(19) United States
(12) Patent Application Publication
(10) Pub. No.: US 2021/0360958 A1
(43) Pub. Date: Nov. 25, 2021

(54) SLEEP-IMPROVING COMPOSITIONS

(71) Applicant: FrieslandCampina Nederland B.V., Amersfoort (NL)

(72) Inventors: Leon Franciscus MALLEE, Wageningen (NL); Anne SCHAAFSMA, Wageningen (NL); Arjen NAUTA, Wageningen (NL); Jouke VELDMAN, Wageningen (NL)

(21) Appl. No.: 17/286,665

(22) PCT Filed: Oct. 1, 2019
(86) PCT No.: PCT/EP2019/076595

§ 371 Date: Apr. 19, 2021

(30) Foreign Application Priority Data
Oct. 17, 2018 (EP) 18200870.6

(51) Int. Cl. A23L 33/21; A23L 33/175; A23L 33/18; A23L 33/19; A23L 33/16; A23L 33/15

(52) U.S. Cl. CPC A23L 33/21; A23L 33/175; A23L 33/15; A23L 33/19; A23L 33/16; A23L 33/18

(57) ABSTRACT

The invention relates to nutritional compositions or nutritional supplements for improving sleep and/or reducing anxiety. Provided is a nutritional composition comprising (i) 1-40 wt %, based on solids content, galacto-oligosaccharides (GOS), and (ii) a protein source providing L-tryptophan (Tip) and L-cysteine (Cys), wherein the protein providing Tip and Cys is present in an amount that provides Cys and Tip each in an amount of 4-15 wt. % relative to the weight of GOS in the composition.

The invention relates to nutritional compositions or nutritional supplements for improving sleep and/or reducing anxiety.

It is well-known that a qualitative sleep is important for brain function, and that sleep can be affected by nutrition and lifestyle. Neurotransmitters play an important role in the processes of sleep and alertness. Although serotonin has been positioned as a sleep inducer for a long time, evidence exists that it plays a role in both sleep and wakefulness, with probably a stronger role in the latter. The indication for this is that stimulation of serotonin transmission, results in quiet non-aroused waking (Miller 2006). On the other hand, melatonin is recognized as a sleep inducing and sleep maintaining hormone, and this hormone needs tryptophan and serotonin as precursors (Brzezinski 1997).

Proteins are the suppliers of dispensable and indispensable amino acids. At least two of the indispensable amino acids, tryptophan and tyrosine, have been linked to sleep and alertness/attention. Both are precursors of neurotransmitters: serotonin and catecholamines, respectively.

Whey proteins have been suggested to be important, since they contain of all milk proteins the highest levels of tryptophan, in particular α-lactalbumin (about 17 wt. % of the whey proteins is α-lactalbumin; see Layman 2018). Although tyrosine levels are slightly lower in bovine whey proteins as compared to caseins, phenylalanine levels are slightly higher and this will compensate the lower tyrosine (Renner 1982). The ratio tryptophan/tyrosine+phenylalanine is most beneficial for α-lactalbumin (0.68) as compared to bovine whey protein in total (0.29) and for that reason, α-lactalbumin maybe might be most effective to promote gastrointestinal health or to modulate neurological function including sleep and depression (Layman 2018, Renner 1982)).

Whey proteins, because of their unique amino acid composition, are furthermore recognized for their ability to optimize aspects of immune function, with perhaps their best-characterized action being stimulation of glutathione and glutamine production. Glutathione (GSH) is synthesized from cysteine, glutamate, and glycine. It plays a major role in protection of cells against oxidative stress. Interestingly, GSH has also been implicated in sleep regulation. Studies in rats have shown that oxidized glutathione (GSSG) significantly enhanced slow wave sleep and paradoxical sleep at the expense of wakefulness during a 12-h dark period, which supports the hypothesis that glutathione may be a sleep-inducing factor in the brain.

Active uptake of tryptophan in the brain is influenced by its plasma concentrations (and thus diet concentrations), and the concentrations of the large neutral amino acids (LNAA) (Young 2005 and 1989). Main competitor is tyrosine, a facultative essential amino acid. Phenylalanine can be easily converted into tyrosine, and should be added to the ‘tyrosine effect’ (Young 2005b). The ratio of the plasma concentrations of tryptophan-other LNAA has proven to be an accurate predictor of changes in brain tryptophan levels (Layman 2018). Markus et al (2005) reported results of a human study wherein participants ingested two night-time protein shakes containing either 20 g of α-lactalbumin (4.8 g of tryptophan per 100 g) or 20 g of casein (1.4 g of tryptophan per 100 g). Two hours after consumption of the second shake, the α-lactalbumin shakes induced increased plasma ratios of tryptophan to LNAAs more than did the casein shakes, and individuals receiving the shake with higher tryptophan content reported less morning sleepiness and improved daily alertness and attention. The authors speculate that this may reflect increased serotonin levels after consumption of the α-lactalbumin shakes. Importantly however, there was no detectable difference in sleep quality or duration between individuals who had consumed α-lactalbumin or casein.

Thus, whereas current data indicate that tryptophan ingestion appears to have beneficial effects on sleep, the potential of dietary proteins to improve sleep remains speculative. As also expressly indicated in the Layman review, further studies should evaluate the potential of habitual dietary intake of dietary proteins like α-lactalbumin to improve sleep conditions, characterizing e.g. the protein dose, adding additional active ingredients, and/or the timing of the meal.

Therefore, the present inventors set out to investigate the potential of whey-derived proteins in sleep-promoting compositions. More in particular, they sought to develop compositions that can reduce anxiety and/or target multiple sleep-related issues in healthy adults, including time to falling asleep, the period of undisturbed sleep, and the feeling of waking up refreshed.

To that end, they elaborated on recent insights that Trp metabolism, leading among others to serotonin in intestinal cells, are under the direct or indirect control of the intestinal microbiota. See Agus et al. (Cell; Vol. 23, issue 6, P716-724, 2018) for recent advances concerning gut microbiota regulation of Trp metabolism in health and disease. The microbiota can directly and indirectly modulate host endogenous Trp metabolism, and variations in Trp metabolism can negatively influence microbial proliferation and microbiota diversity. Reports have shown (reviewed by Gao et al., Front Cell Infect Microbiol. 2018; 8: 13) that the bacterial community can influence Trp metabolism and the serotonergic system. The balance between bacterial Trp metabolism and Trp synthesis determines local GI and circulating Trp availability for the host.

Interestingly, specific gut bacteria determine the availability of Trp to the host and then regulate serotonin and subsequent melatonin synthesis (Wikoff et al., (2009) Proc Natl Acad Sci USA. 10;106(10):3698-703. 2009). Furthermore, it has been revealed that the gut microbiota can also regulate the glutathione metabolism of the host, at least shown in mice (Mardinoglu et al., Molecular Systems Biology, 2015; 11 (10)).

It is well known that prebiotics can beneficially affect host health by selectively stimulating the growth and/or activity of gut bacteria. For example, the non-digestible carbohydrate GOS, a typical prebiotic, has been demonstrated to promote growth of various bacterial species, including L. reuteri (Ben et al. World J Gastroenterol 2008;14(42):6564-6568). Therefore, the present inventors hypothesized that sleep-promoting effects can be obtained upon ingestion of dietary protein rich in Trp and Cys, combined with a prebiotic that can promote metabolism of these amino acids into sleep-enhancing components.

It was surprisingly found that beneficial effects on the duration and quality of sleep in human adults are achieved with a nutritional composition comprising (i) 1-40 wt %, based on solids content, of galacto-oligosaccharides (GOS), and (ii) a protein source providing L-tryptophan (Trp) and L-cysteine (Cys), wherein the protein providing Trp and Cys is present in an amount that provides Cys and Trp each in an amount of about 4-15 wt. % relative to the weight of GOS in the composition. Very good results were obtained upon ingestion of a composition providing 0.3 to 0.6 gram of Trp, about 0.3 to 0.6 gram of Cys and about 3.5 to 8 gram of GOS prior (e.g. 1-2 hours) to bedtime.

Sleep-promoting nutritional compositions comprising GOS are known in the art. WO2010/060722 relates to the use of a probiotic bacterial strain in the manufacture of a medicament or therapeutic nutritional composition for improving the maturation of sleep patterns in infants, young children or young animals and/or for reducing sleep disturbances and/or improving sleep patterns in humans or animals. The composition may further contain a prebiotic component in an amount of 0.3 to 10%. According to WO2010/060722, the type of protein is not believed to be critical provided that the minimum requirements for essential amino acid content are met and satisfactory growth is ensured. Although it is preferred that over 50% by weight of the protein source is whey, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in whatever proportions are desired. Hence, nothing in WO2010/060722 hints at selecting Trp/Cys-rich proteins, let alone to combine such proteins in the specific amount relative to GOS.

In a preferred embodiment of the present invention, the protein source providing Trp and Cys is present in an amount that provides Cys and Trp each in an amount of about 7-13 wt %, preferably 8-12 wt %, relative to the weight of GOS in the composition.

As will be understood, in order to deliver a sufficient amount of “sleep-promoting” amino acids, the use of a protein source that is relatively rich in Trp and Cys is advantageously used. In one embodiment, the protein source contains Trp and Cys each in an amount of at least 4 wt. %, preferably at least 4.5 wt. %. Whereas as there is no preferred upper limit, the Trp and/or Cys content of the protein source for use in the present invention will typically be not more than about 8 wt. %. In a specific aspect, the Trp and Cys content are about equal. The protein source may be an intact, naturally occurring protein, a protein hydrolysate, free amino acids or any combination thereof. In one embodiment, intact protein is used. In another embodiment, a protein hydrolysate is used. In still another embodiment, free amino acids are used.

Combinations of two or more protein sources rich in Trp and Cys are also envisaged. It is however preferred that the nutritional composition does not contain a significant amount of proteins or protein hydrolysates that are relatively poor in Trp and Cys, since this will “dilute” the effect of amino acids provided by the sleep-promoting protein source(s). Typically, the Trp/Cys providing protein source (e.g. with a Trp and Cys content of at least 4 wt. %) is the predominant proteinaceous source in the composition, accounts for at least 70 wt. %, preferably at least 80 wt. %, more preferably at least 90 wt. % of the total protein fraction present in the composition. In a preferred embodiment, the Trp/Cys-rich protein source(s) is/are the sole protein source in the nutritional composition.

In one embodiment, the egg protein lysozyme which contains about 8 wt. % Trp and 6.4 wt. % Cys, is used as Trp/Cys providing protein source

A very suitable protein source for use in a sleep-promoting composition is whey-derived protein, in particular alpha-lactalbumin (aLac) having a Trp content of 4.8 wt. % and a Cys content of 4.8 wt. %.

aLac is found in bovine milk at approximately 1.5 g/l, and it is the second most abundant protein in whey, accounting for approximately 20% w/w of the total whey proteins. The molecular weight of aLac 14 175 Da, and three genetic variants of aLac have been identified. aLac can be isolated from whey by ultrafiltration followed by selective acid precipitation; ion-exchange chromatography or tryptic digest of contaminating proteins followed by ultrafiltration. A number of companies manufacture and supply alpha-lactalbumin including Arla Foods (Denmark), Amor Proteins (France) and Agropur (Canada).

In one embodiment, the protein source in a sleep-promoting composition provided herein comprises or consists of a whey protein isolate (WPI) that is enriched in aLac, preferably a WPI having an aLac content of at least 40 wt. %/protein or a WPC (whey protein concentrate), preferably enriched to at least 40 wt. % alpha lac/protein. The WPC may be a WPC having a protein content of between 30-82 wt. % on dry matter. The WPC may be a WPC 30, 35, 60 or 80. Preferably, the WPC is derived from acid whey, cheese whey or milk microfiltration permeate.

A nutritional composition of the invention is characterized among others in that the Trp/Cys-providing protein source is combined with GOS. GOS are carbohydrate components that are not digestible by humans, but which have been shown to have a growth-promoting effect on bifidobacteria and lactobacilli, as they are able to ferment them. Typically, the basic structure of GOS includes a lactose core at the reducing end which is elongated typically with up to seven galactose residues (degree of polymerization of 8; DP8). In the past decade, GOS have had an increasing application in human food products, including dairy products, sugar replacements and other nutritional or nutraceutical supplements. However, it has heretofore never been used in a specific weight ratio to Trp/Cys-rich protein sources, let alone for use of the resulting combination as sleep-promoting or sleep-supporting agent.

The nutritional composition according to the present invention comprises 1-40 wt %, preferably 5-40 wt %, more preferably 10-40 wt %, and most preferably 10-30 wt % of galacto-oligosaccharides (GOS). These weight percentages are based on total solids content of the composition.

Typically, a composition of the invention is formulated to provide a daily amount of 3.5 to 10 grams of GOS, which may be divided over one or more servings. For example, a serving of a sleep-promoting nutritional composition may contain 2-10 g GOS, preferably 3-8 g GOS, more preferably 4-6 g GOS. As used herein, the term “serving” refers to a portion of about 15-50 grams, or about 150-500 mL. In a preferred aspect, the composition is formulated to provide about 0.3 to 0.6 gram of Trp, about 0.3 to 0.6 gram of Cys and about 3.5 to 8 gram of GOS per serving.

GOS can be produced by known chemical methods, but the preferred method to synthesize them is the enzymatic approach. Commercial GOS preparations are generally produced via a transgalactosylation reaction by enzymatic treatment of lactose with β-galactosidases (EC.3.2.1.23) from different sources such as fungi, yeast and/or bacteria, yielding a mixture of oligomers with varied chain lengths, resulting in the formation of a mixture containing approximately 100 different types structures with varying DP and linkages. Beta-Galactosidase is produced in many microorganisms such as Bacillus circulans, Aspergillus oryzae, Kluyveromyces marxianus, Kluyveromyces fragilis, Sporobolomyces singularis, and Lactobacillus fermentum.

A composition provided herein may contain additional nutritional agents, such as vitamins, minerals and/or biologically active peptides. In one embodiment, the composition comprises vitamins and minerals, preferably one or more of magnesium, zinc, vitamin B3 and vitamin B6 in an amount that can support or promote sleep. In a specific embodiment, it comprises magnesium, zinc, vitamin B3 and vitamin B6.

The vitamins and minerals may be present in any effective relative or absolute amount. Vitamin B3 may be present at about 0.025 to 0.1 wt. % of the total dry weight of the composition. Vitamin B6 may be present at about 0.002 to 0.01 wt. % of the total dry weight of the composition. Good results are obtained when vitamin B3 and vitamin B6 are used in a relative weight ratio in the range of 20:1 to 1:1, preferably 15:1 to 5:1. For example, the nutritional composition is formulated to provide a daily amount (divided over e.g. one or two dosage units) of about 5-15 mg vitamin B3 and about 0.5 to 2 mg vitamin B6. In a specific aspect, a sleep-promoting composition comprises about 10 mg vitamin B3 and about 1 mg vitamin B6 per serving.

Magnesium may be present at about 0.5 to 1.5 wt. % of the total dry weight of the composition. In a specific aspect, a single serving size of the sleep-promoting composition comprises about 100-300 mg magnesium 10 mg vitamin B3 and about 1 mg vitamin B6.

Zinc may be present at about 0.01 to 0.05 wt. % of the total dry weight of the composition. In a specific aspect, a serving size of the sleep-promoting composition comprises about 2 to 15 mg zinc, preferably about 2 to 10 mg zinc, and most preferably about 5 mg zinc, per serving.

In addition to the Cys/Trp-providing source of (intact) protein, the composition may comprise one or more a sleep-promoting bioactive peptide(s), e.g. a polypeptide up to about 20 amino acids in length, which exerts its action independent of providing the Cys and Trp amino acids as precursors for sleep-enhancing metabolites. Preferably, said additional peptide is present in an amount of about 0.1 to 10 weight %, more preferably about 0.5-5 weight %, based on the total weight of GOS in the composition. In one embodiment, a serving of the sleep-promoting composition comprises about 100-400 mg sleep-promoting bioactive peptide(s). For example, the sleep-promoting milk derived peptide is a sleep-promoting milk-derived peptide, preferably a peptide resulting from tryptic digestion of casein. In one embodiment, it is an aS1-casein tryptic hydrolysate containing a bioactive peptide that acts on GABA receptors. In a specific aspect, the polypeptide comprises or consists of the amino acid sequence Tyr—Leu—Gly—Tyr—Leu—Glu—Gln—Leu—Leu—Arg. This decapeptide is marketed under the tradename Lactium™ (Ingredia, France).

Unlike the compositions disclosed in WO2010/060722 for improving sleep in infants, young children or young animals, a sleep-promoting or anxiety-reducing nutritional composition or supplement of the present invention does not require, or rely on, the administration of probiotics. Accordingly, in one embodiment a composition provided herein does not comprise a probiotic bacterial strain.

A nutritional composition provided herein may contain a flavoring agent, a preservative and/or a coloring agent.

A nutritional composition of the invention can be formulated in any desirable form. For example, the composition may be in a dry, liquid or semi-liquid state. In a preferred embodiment, unit dosage forms of the compositions are consumed orally, including tablets, capsules, pills, lozenges, wafers, powders, liquids, emulsions, suspensions, solutions and the like. The composition can have the form of or can be included in a food or a beverage, such as a dietary supplement bar or shake.

Preferred formulations include a liquid suspension, a powder (e.g. to be reconstituted with water, juice, milk, or any other suitable beverage prior to use) or a tablet. As used herein, the term “beverage” or “drink” refers generally to any liquid or semi-solid form suitable for oral consumption by an individual that is now known or becomes known by those skilled in the art, and also includes concentrates, for example, frozen concentrates, and freeze-dried powders that can be dissolved in a suitable volume of liquid carrier to generate a, so called, “instant” liquid or semi-solid for consumption by an individual. For example, when administered in the form of a beverage, compositions of the present invention may be water-based, milk-based, tea-based, fruit juice-based, or some combination thereof. In certain embodiments the nutritional drink of the invention have a viscosity of from about 0.3×10−3 Pa·s to about 1.0 Pa·s.

As will be appreciated by a person skilled in the art, a composition according to the invention has many interesting applications, in particular for use as sleep-improving agent and/or as a stress-reducing or anxiety-reducing agent.

In one embodiment, the invention provides a nutritional composition comprising (i) galacto-oligosaccharides (GOS), and (ii) a protein source providing L-tryptophan (Trp) and L-cysteine (Cys), wherein the protein providing Trp and Cys is present in an amount that provides Cys and Trp each in an amount of about 4-15 wt. % relative to the weight of GOS in the composition, for use in a method of supporting or enhancing at least one, preferably at least two or more, qualitative and/or quantitative aspect(s) of sleep in a subject. In one aspect, supporting or enhancing qualitative and/or quantitative aspect(s) of sleep comprises one or more of the following:
(i) promoting falling asleep;
(ii) inducing or supporting a mature sleep pattern;
(iii) reducing or preventing sleep disturbances/sleeping more time while in bed;
(iv) increasing the feeling of a deep sleep;
(v) feeling more refreshed at waking up;
(vi) feeling more energized and/or having a better mood during daytime.

The nutritional composition is preferably used so that in a daily dose 0.3 to 0.6 gram of Trp and about 0.3 to 0.6 gram of Cys is consumed.

The use of the nutritional composition is preferably carried out so that the GOS in a daily dose of 3.5 to 11 gram, preferably 4 to 8 gram, is consumed.

The use of the nutritional composition is preferably carried out so that the optional sleep-promoting polypeptide, e.g. a tryptic casein digest, in a daily dose of 100 to 1000 mg, preferably 150 to 500 mg, is consumed.

Provided that the composition contains magnesium, the use of the composition is preferably carried out so that magnesium in a daily dose of 40 mg to 500 mg, preferably 150 mg to 250 mg is received.

Provided that the composition contains zinc, the use of the composition is preferably carried out so that zinc in a daily dose of 0.5 mg to 20 mg, preferably 1 mg to 10 mg is received.

Provided that the composition contains vitamin B3, the use of the composition is preferably carried out so that vitamin B3 in a daily dose of 2 mg to 30 mg, preferably 5 mg to 15 mg is received.

Provided that the composition contains vitamin B6, the use of the composition is preferably carried out so that vitamin B6 in a daily dose of 0.2 mg to 10 mg, preferably 0.5 mg to 3 mg is received.

Also provided is a method for supporting or enhancing at least one, preferably at least two or more, qualitative and/or quantitative aspect(s) of sleep in a subject, comprising administering to the subject a nutritional composition as herein disclosed. The present invention encompasses unique compositions and methods of treating or preventing insomnia or sleeplessness, improving daytime energy and/or promoting nighttime relaxation. The methods comprise the administration of the unique compositions of the present invention, comprising a combination of sleep-promoting agents of different nature (proteins and oligosaccharides) which, when used a specific weight ratio, work synergistically to shorten the time needed to fall asleep and improve the quality of sleep by reducing the number of awakenings during sleep and/or increasing the duration of sleep. Without wishing to be bound by theory, through this coordinated combination of nutrients, regeneration during sleep (sleep recovery processes) is promoted and eventual negative effects of lack of sleep are reduced. The present invention affords a safe and natural way of supporting or improving sleep quality without the use of hormones or pharmaceutical sedatives.

Typically, the subject is a human subject. Preferably, it is a human subject of at least 4, more preferably at least 6, even more preferably at least 12, more preferably at least 16, and most preferably at least 18 years old. Most preferably, it is an adult or elderly human subject. In another embodiment, it is a sportsperson or a student. In a specific embodiment, the subject is not taking any other (e.g. conventional) sleep-improving agents, such as prescribed sleep medication. Also, the subject is preferably not known or suspected of having a cognitive dysfunction and/or emotional disturbances occurring in neuropsychiatric illnesses or disorders, or in aging. In one embodiment, a composition herein disclosed finds its use in optimizing the nocturnal rest and sleep quality and sleep efficiency of normal sleepers (sleeping healthy people).

The dosage forms of the composition provided herein are formulated for administration at various rates. Administration of one dosage form prior to a desired period of sleep is preferred. For example, preferred is the evening intake, e.g. by oral ingestion, of the nutritional composition or dietary supplement. In one embodiment, the composition is consumed for example, 120 minutes, 60 minutes or 30 minutes until immediately before going to bed.

Basically, the dietary supplement is suitable both for people with sleep disorders as well as for persons without chronic sleep problems (“sleep-healthy” people). Since the dietary supplement, as explained above, accelerates the processes occurring during sleep regeneration processes and optimized and thus intensified the sleep or the effected by the sleep phase relaxation effect, it is particularly suitable for those not suffering from morbid sleep, but a lack of sleep (for example, have due to occupational exposure, etc.) are exposed to a sleep deprivation, and/or have an increased need for sleep. With lack of sleep, a sleep duration is meant less than the average sleep requirement of the persons concerned.

### LEGEND TO THE FIGURES

FIG. 1. The mean total PSQI scores and SD over the 21-day. Divided into all participants included, participants without sleep medication and participants using sleep medication during 21-day challenge. *significant decrease of PSQI score.

FIG. 2. The mean total VAS A scores (feeling rested/refreshed) and SD over the 21-day test period. Divided into all participants included, participants without sleep medication and participants using sleep medication during 21-day challenge. *significant increase

FIG. 3. The mean total VAS B (mood) scores and SD over the 21-day test period. Divided into all participants included, participants without sleep medication and participants using sleep medication during 21-day challenge. *significant increase

FIG. 4. The mean total fit scores and SD over the 21-day test period. Divided into all participants included, participants without sleep medication and participants using sleep medication during 21-day challenge. *significant increase

### EXPERIMENTAL SECTION

The following Examples 1-4, exemplify a nutritional composition according to the invention for use among others in the sleep optimization, the reduction of anxiety, the improvement of sleep specific regeneration and/or as natural sleep aid.

### EXAMPLE 1

### Nutritional Composition

| | Name | Amount | |
|---|---|---|---|
| Total amount | | 16.3 | g |
| Ingredients: | Alpha-lactalbumine* | 10 | g |
| | GOS** | 5 | g |
| | Lactium*** | 300 | mg |
| | Niacin | 10 | mg |
| | Vitamin B6 | 1.0 | mg |
| | Magnesium | 200 | mg |
| | Zinc | 5 | mg |

*Agropur, Canada
**Vivinal GOS powder (GOS content 77 w %)
***Ingredia, France

### EXAMPLE 2

### Alternative Nutritional Composition

| | Name | Amount | |
|---|---|---|---|
| Total amount | | 16.3 | g |
| Ingredients: | Whey Protein Isolate* | 10 | g |
| | GOS** | 5 | g |
| | Lactium | 300 | mg |
| | Niacin | 10 | mg |
| | Vitamin B6 | 1.0 | mg |
| | Magnesium | 200 | mg |
| | Zinc | 5 | mg |

*FrieslandCampina The Netherlands
**Vivinal GOS powder (GOS content 77 w %)

### EXAMPLE 3

### Alternative Nutritional Composition

| | Name | Amount | |
|---|---|---|---|
| Total amount | | 16.3 | g |
| Ingredients: | Whey Protein hydrolysate* | 10 | g |
| | GOS** | 5 | g |
| | Lactium | 300 | mg |
| | Niacin | 10 | mg |
| | Vitamin B6 | 1.0 | mg |
| | Magnesium | 200 | mg |
| | Zinc | 5 | mg |

*Hyvital ETD 120, (WPC 80), FrieslandCampina The Netherlands
**Vivinal GOS powder (GOS content 77 w %)

### EXAMPLE 4

### Alternative Nutritional Composition

| | Name | Amount | |
|---|---|---|---|
| Total amount | | 11.3 | g |
| Ingredients: | Tryptophan rich peptides* | 2 | g |
| | Cysteine rich peptides** | 3 | g |
| | GOS*** | 5 | g |
| | Lactium | 300 | mg |
| | Niacin | 10 | mg |
| | Vitamin B6 | 1.0 | mg |
| | Magnesium | 200 | mg |
| | Zinc | 5 | mg |

*prepared according WO 02/46210
**prepared according EP1201137
***Vivinal GOS powder (GOS content 77 w %)

### EXAMPLE 5

### Consumer Study

### Materials and Methods

### Study Design

This Example describes an uncontrolled consumer study to investigate the effect of the daily intake of a nutritional test composition on various aspects of sleep in human adult volunteers (see Scheme 1). The exclusion criteria were lactose intolerance and pregnancy. Potential participants were identified via a special Facebook account for people with sleep disturbances. The consumer study was initiated by and monitored at the FrieslandCampina Innovation Centre in Wageningen between June 2018 and September 2018. Participants were able to perform the 21 days challenge at home. Participants were not restricted in any kind of medication or to behavior that is known to influence sleep.

Assessments of sleep quality, mood, stress and feeling fit were completed by the participants via an online questionnaire at baseline (day 0), and after 7, 14 and 21 days. The Visual Analogue Scale (VAS) was completed via an offline questionnaire. All assessments were based on subjective questionnaires.

### Study Treatment

The test product was a powder blend having the composition shown in Example 1, but without zinc. The test product was supplied as 21 sachets+one spare sachet. The participants were asked to take one sachet daily, reconstitute the powder with 150 ml water, stir it, and consume the reconstituted product shortly, preferably 1 h, before going to bed.

### Primary and Secondary Outcome Measurements

The primary outcome was the change in overall sleep quality after 21 days of daily consumption of the test blend and measured by the Pittsburgh Sleep Quality Index (PSQI) (Buysse, D. J. et al. (1989). Psychiatry research, 28(2), 193-213). The PSQI is a self-rated questionnaire with 19 questions divided into seven “component” scores, each of which has a range of 0-3 points. In all cases, “0” indicates no difficulty, and “3” indicates severe difficulty. Altogether the score has a range of 0-21 points, “0” indicates no difficulty and “21” indicates severe difficulty in all areas. The items include hours of sleep, ratings for frequency of sleep concerns, general sleep quality and daytime factors related to poor sleep. The Secondary outcomes (on a scale of 0 to 10) were “wake up fresh” (VAS A) and “mood” (VAS B) and “experience of feeling fit”.

### Statistical Analysis

Data were analyzed using IBM SPSS Statistics (version 24, IBM Corp., Armonk, USA). A generalized estimating equations (GEE) was used to analyze the change of the PSQI, VAS A, VAS B, and the fit score over time. The level of significance was set at 5%. Gender, age and sleep medication (component 6 of the PSQI) were included as confounding variables. Time in days was included in the analysis as a continuous or categorical variable.

### Results

### Participant Characteristics

From a total of 127 participants, 89 (44 males and 45 females) were eligible for the consumer study. The other 38 participants were excluded for the 21 day challenge based on lactose intolerance. 76 participants started with the 21 days challenge 36 males and 40 females. Some participants dropped-out of which 5 stopped due to disliking the product, feeling nauseous or headache after consuming the product. Others did not complete the final questionnaires. No serious adverse events reported during the 21-day treatment period. 50 participants (23 males, 27 females) finished the 21 days challenge and filled in the online questionnaires. The participants' baseline characteristics are shown in Table 1.

TABLE 1
Participant characteristics included
in evaluation (population; n = 50)

| Characteristics | |
|---|---|
| Male (M), n (%) | 23 (46%) |
| Female (F), n (%) | 27 (54%) |
| Age group | |
| 25-44 | N = 20 (F = 11, M = 9) |
| 45-65 | N = 30 (F = 16, M = 14) |
| Sleep medication during | 8 (16%) |
| challenge n, (%) | |
| Medication during challenge | |
| 25-44 | N = 3 (F = 2, M = 1) |
| 45-65 | N = 5 (F = 3, M = 2) |

### Primary Outcome

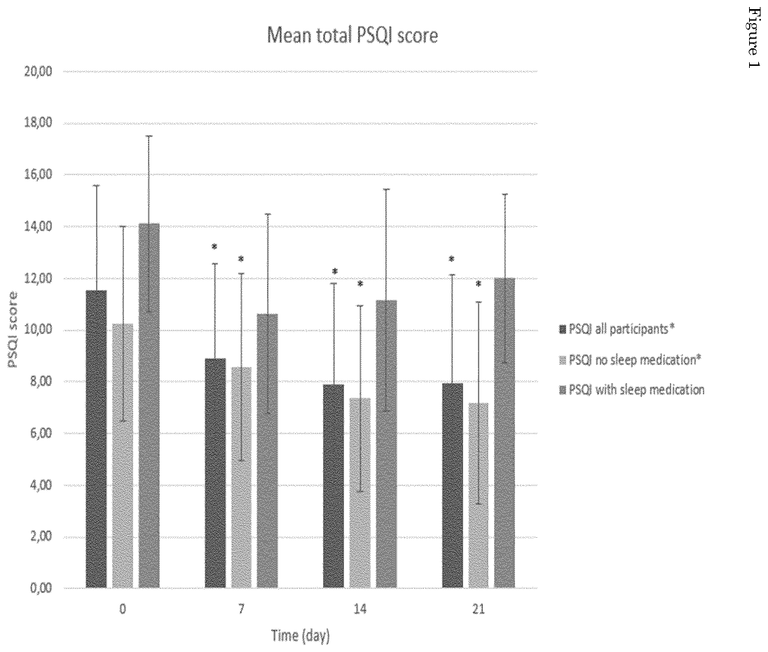

FIG. 1 shows the mean change in total PSQI during the overall study. Taking into account the results of all participants, a significant reduction (from 11.56⊥4.05 to 7.96⊥4.17, p<0.000) was observed in mean total PSQI score from baseline to end of treatment (day 21) (see also Table 2). A lower PSQI means an improvement of sleep quality. The total PSQI reduced significantly between day 0 and day 7 (p<0.0001) and day 0 and day 14 (p<0.000). The largest significant decrease of PSQI score was found between day 0 and day 14 (from 11.56±4.05 to 7.91±3.90). No significant difference was found between day 14 (7.91±3.90) and day 21 (7.96±4.17) (p=0.717).

The GEE analyzed the influence of the confounders age, gender and use of sleep medication. A reduction of the PSQI score was found in the age group 25-44 years (from 11.00±3.85 to 7.55±4.54) and 45-65 years (from 11.93±4.19 to 8.23±3.97) (p=0.332). Also, a reduction of the PSQI score was found between females (from 12.07±3.84 to 7.70±3.74) and males (from 10.96±4.28 to 8.26±4.70) (p=0.880). Based on these results it can be concluded that the formula is suitable for males, females and different age groups.

When the effect of using sleep medication on the PSQI scores was taken into account, a difference was observed between participants using sleep medication and participants without the use of sleep medication (p<0.000). In participants without sleep medication, the PSQI score was reduced from 10.24±3.74 to 7.19±3.90, p<0.000 over the course of the study. In contrast, no significant overall reduction of the PSQI score was found in participants on sleep medication (from 14.12±3.41 to 12.00±3.25, p=0.054). Only between day 0 and day 7, the PSQI score dropped significantly (from 14.12±3.41 to 10.63±3.85, p<0.004).

TABLE 2
Overview mean scores of the different outcome measurements

| | | | |
|---|---|---|---|
| | All participants | No sleep | With sleep |
| Outcome | included | medication | medication |
| measure | (mean (SD)) | (mean (SD)) | (mean (SD)) |

| | | | | | | |
|---|---|---|---|---|---|---|
| PSQI | | | | | | |
| Day 0 | 11.56 | (4.05)* | 10.24 | (3.74)* | 14.12 | (3.41) |
| Day 7 | 8.88 | (3.70)* | 8.55 | (3.62)* | 10.63 | (3.85)* |
| Day 14 | 7.91 | (3.90)* | 7.35 | (3.59)* | 11.14 | (4.30) |
| Day 21 | 7.96 | (4.17)* | 7.19 | (3.90)* | 12.00 | (3.25) |
| VAS A | | | | | | |
| Day 0 | 4.35 | (1.96) | 4.46 | (2.18) | 4.23 | (1.83) |
| Day 7 | 5.03 | (1.72) | 4.75 | (1.66) | 5.64 | (1.82) |
| Day 14 | 5.69 | (1.63)* | 5.51 | (1.70)* | 5.95 | (1.75)* |
| Day 21 | 6.28 | (1.75)* | 6.33 | (1.97)* | 6.10 | (0.80)* |
| VAS B | | | | | | |
| Day 0 | 5.69 | (2.16) | 5.11 | (2.40) | 6.34 | (1.78) |
| Day 7 | 6.01 | (1.57) | 5.90 | (1.68) | 6.26 | (1.43) |
| Day 14 | 6.42 | (1.56)* | 6.33 | (1.74)* | 6.58 | (1.35)* |
| Day 21 | 6.72 | (1.45)* | 6.84 | (1.62)* | 6.35 | (0.70)* |
| Fit score | | | | | | |
| Day 0 | 5.89 | (1.41) | 5.95 | (1.39) | 5.76 | (1.47) |
| Day 7 | 5.96 | (1.50) | 6.08 | (1.31) | 5.31 | (2.25) |
| Day 14 | 6.61 | (1.24)* | 6.62 | (1.29)* | 6.57 | (0.98)* |
| Day 21 | 6.85 | (1.24)* | 6.95 | (1.26)* | 6.38 | (1.06)* |

Note:
*p < .05.

### Secondary Outcomes

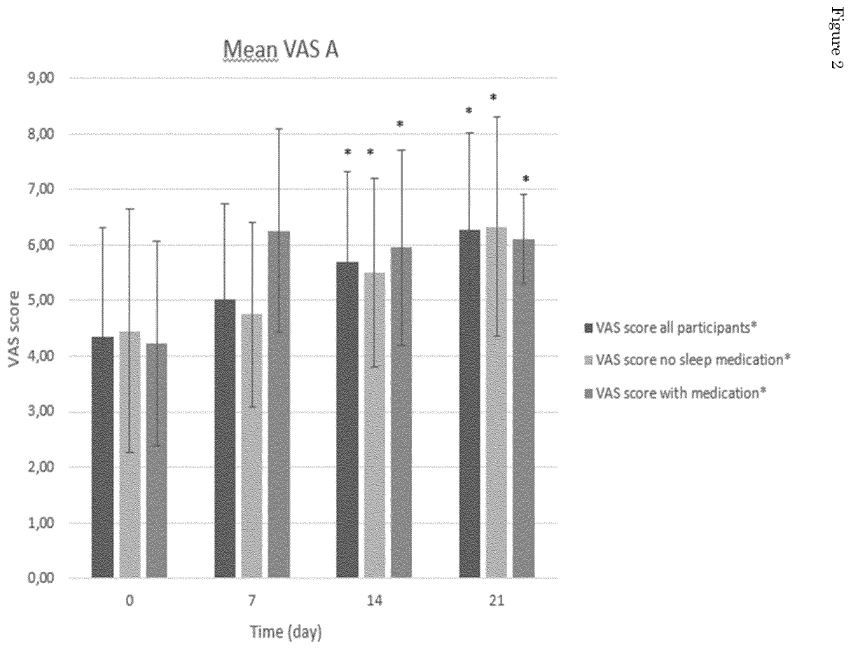

Only 17 participants (8 males, 9 females) had completed the VAS A and VAS B questionnaires . The characteristics of these participants are shown in table 4. As illustrated in FIG. 2, the mean VAS A score was significantly higher after 14 days (p<0.003) and 21 days (p<0.003). The overall increase was from 4.35 ±1.96 to 6.28 ±1.75 over 21 days (p<0.000) (see table 3), indicating a better mood after waking up after 14 and 21 days of taking the test composition. No significant differences were found for age (p=0.193), gender (p=0.140) and medication (p=0.232).

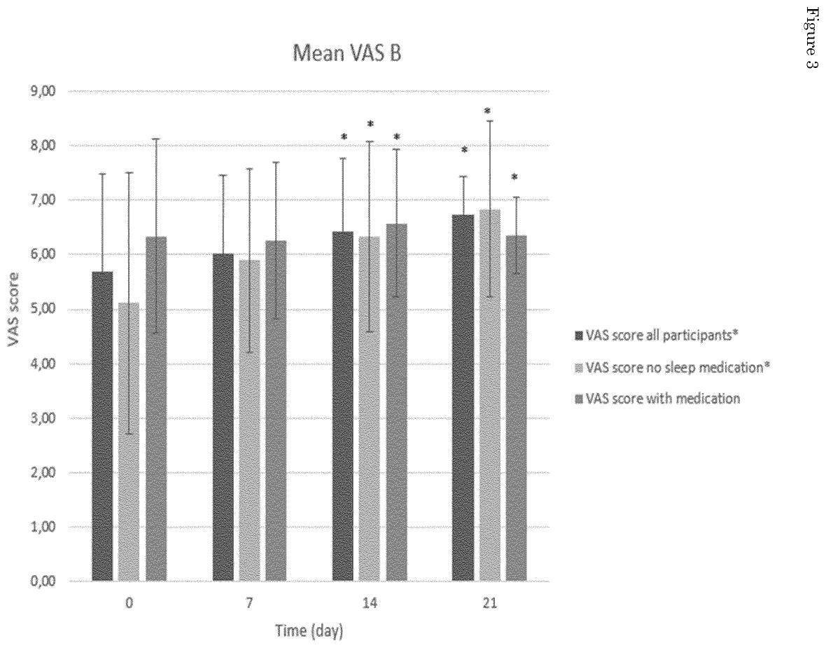

The mean scores of VAS B are illustrated in FIG. 3. The mean VAS B score was significantly increasing (p<0.009) over the 21 days (see table 2). An improvement of the VAS B score was found between day 0 and day 14 from 5.69±2.16 to 6.42±1.45 (p<0.031). Overall the increase was from 5.69±2.16 to 6.72±1.45 over 21 days, indicating a better mental well-being after taking the sachets for 14 and 21 days. No significant differences were found for gender (p=0.975), age (p=0.913) and medication (p=0.133) (see table 2).

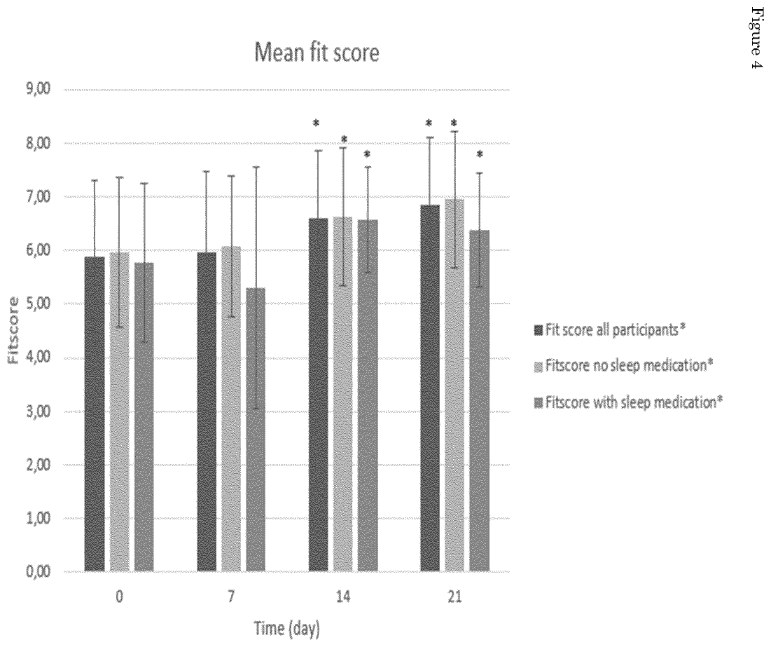

The experience of feeling fit was evaluated by all 50 participants via an online questionnaire. The mean scores are illustrated in FIG. 4.

A significant improvement of the fit score was found over the 21 days (6.85±1.24) compared with the baseline measurement (5.89 ±1.41) (p <.000) (see table 3). The participants felt fitter over the 21 days treatment period. No significant difference was found between day 0 and day 7 (p=0.739), but there is a significant improvement between day 0 and day 14 (p<0.000). No significant differences were found between gender (p=0.545), age (p=0.583) and medication (p=0.122).

TABLE 3
Participants' characteristics VAS questionnaire (n = 17)

| Characteristics | |
|---|---|
| Male (M), n (%) | 8 (47%) |
| Female (F), n (%) | 9 (53%) |
| Age group | |
| 25-44 | N = 6 (F = 3, M = 3) |
| 45-65 | N = 11 (F = 6, M = 5) |
| Sleep medication during | 5 (29%) |
| challenge n, (%) | |
| Medication during challenge | |
| 25-44 | N = 1 (F = 1, M = 0) |
| 45-65 | N = 4 (F = 2, M = 2) |

In conclusion, this consumer test indicates a beneficial effect of the formula on sleep quality as measured by PSQI score in apparently healthy human subjects not taking conventional sleep medication.

1. A nutritional composition comprising (i) 1-40 wt % galacto-oligosaccharides (GOS) based on solids content, and (ii) a protein source providing L-tryptophan (Trp) and L-cysteine (Cys), wherein the protein source providing Trp and Cys is present in an amount that provides Cys and Trp each in an amount of 4-15 wt. % relative to the weight of GOS in the composition.

2. The nutritional composition of claim 1, wherein the protein source providing Trp and Cys is present in an amount that provides Cys and Trp each in an amount of 7-13 wt. %, preferably 8-12 wt. %, relative to the weight of GOS.

3. The nutritional composition of claim 1, wherein the protein source contains Trp and Cys each in an amount of at least 4 wt. %.

4. The nutritional composition of claim 1, wherein the protein source in the composition comprises or consists of a whey-derived protein and/or lysozyme.

5. The nutritional composition of claim 4, wherein said whey-derived protein comprises or consists of a whey protein isolate (WPI) or WPC (whey protein concentrate) that is enriched in alpha-lactalbumin (aLac), preferably wherein said whey-derived protein is aLac.

6. The nutritional composition of claim 1, comprising 5-40 wt %, preferably 10-40 wt %, preferably 10-30 wt % GOS, based on solids content.

7. The nutritional composition of claim 1, wherein said composition further comprises one or more of magnesium, zinc, vitamin B3 and vitamin B6, preferably wherein said composition comprises magnesium, zinc, vitamin B3 and vitamin B6.

8. The nutritional composition of claim 1, wherein said composition further comprises a peptide resulting from tryptic digestion of casein, preferably in an amount of 1 to 10 weight %, more preferably 3-8 weight %, based on the total weight of GOS in the composition.

9. The nutritional composition of claim 1, wherein said composition is in a dry, liquid or semi-liquid state.

10. A method of improving sleep, reducing stress, and/or reducing anxiety in a subject, the method comprising administering to the subject an effective amount of the nutritional composition of claim 1.

11. A method of supporting or enhancing at least one, preferably at least two or more, of the following qualitative and/or quantitative aspect(s) of sleep in a subject:
(i) promoting falling asleep;
(ii) inducing or supporting a mature sleep pattern;
(iii) reducing or preventing sleep disturbances/sleeping more time while in bed;
(iv) increasing the feeling of a deep sleep;
(v) feeling more refreshed at waking up; and
(vi) feeling more energized and/or having a better mood during daytime, the method comprising administering to the subject an effective amount of the nutritional composition of claim 1.

12. The method of claim 11, wherein the subject is a human subject, preferably an adult subject, more preferably an adult subject not taking any further sleep-improving agents.

13. The method of claim 11, wherein the effective amount of the composition administered to the subject provides 0.3 to 0.6 gram of Trp, 0.3 to 0.6 gram of Cys, and 3.5 to 8 gram of GOS daily, preferably as a single serving.

14. The method of claim 11, wherein the composition is administered to the subject within 120 minutes, preferably within 90 minutes, prior to bedtime.
